(19) United States
(12) Patent Application Publication
(10) Pub. No.: US 2025/0129320 A1
(43) Pub. Date: Apr. 24, 2025

(54) SCALABLE SYSTEMS AND METHODS FOR CONTINUOUS TRANSFECTION OF CELLS

(71) Applicant: Life Technologies Corporation, Carlsbad, CA (US)

(72) Inventor: Xavier De Mollerat Du Jeu, Encinitas, CA (US)

(21) Appl. No.: 18/689,952

(22) PCT Filed: Oct. 7, 2022
(86) PCT No.: PCT/US2022/046107

§ 371 Date: Mar. 7, 2024

Related U.S. Application Data
(60) Provisional application No. 63/255,357, filed on Oct. 13, 2021.

(51) Int. Cl. C12M 1/00; C12M 1/06; C12M 1/36; C12N 15/86

(52) U.S. Cl. CPC C12M 29/06; C12M 27/02; C12M 41/48; C12N 15/86; C12N 2740/15043; C12N 2750/14143

(57) ABSTRACT

Methods and systems for improving the scalability and associated efficacy of continuous transfection processes, such as continuous transient transfection processes. The continuous transfection system includes at least a first input line and a second input line, wherein the first input line and the second input line are configured for sterile, fluid connection to a first reagent housing and a second reagent housing, respectively, a mixing chamber that is fluidly connected to the at least first and second input lines, a delivery line fluidly connected to the mixing chamber, wherein the delivery line is configured for sterile, fluid connection to a cell culture vessel, and a flow rate control component, such as a pump operable to control flow rate of liquid into the at least first and second input lines, throughout the mixing chamber, and into the cell culture vessel.

### CROSS REFERENCE TO RELATED APPLICATION(S)

This application claims the benefit of priority to U.S. Provisional Application No. 63/255,357, filed Oct. 13, 2021, the disclosure of which is considered part of, and is incorporated by reference in the disclosure of this application.

### BACKGROUND

### Field

The present invention relates generally to molecular biology, and more particularly to scalable systems and methods for transfecting cells.

### Background Information

Various methods are known and used to introduce biomolecules (e.g., nucleic acids, proteins, ribonucleoproteins, etc.), into cells, including electroporation, microinjection, lipid-mediated transfection, polymer-mediated transfection (e.g., polyethyleneimine/PEI-mediated transfection), viral-mediated transfection, and the like.

Non-mechanical transfection methods typically involve complexation of a payload with a transfection reagent (e.g., a cationic/ionizable lipid transfection reagent, a polymeric transfection reagent, or the like) for a certain duration of time to create a transfection complex. Target cells are subsequently contacted with the transfection complex, which interacts with a cell membrane (e.g., via endocytosis, fusion, or the like). This ultimately results in the internalization and delivery of the payload to the interior of the cell. Known parameters which affect transfection efficiency include, for example, transfection complexation volume, complexation time, extent of exposure of the transfection complex to cells, as well as cell health, cell density and the like.

Systems that enable consistent, continuous formation of transfection complexes (e.g., at a predetermined ratio) and enable consistent and continuous exposure of cells to transfection complexes (e.g., at a predetermined time, flow rate, temperature, and reagent mixture following transfection complex formation), are highly desirable. Automated systems that provide continuous transfection in a scalable manner with minimal user intervention thus solve an unmet need.

### SUMMARY

In various aspects, the present disclosure generally relates to systems and methods for continuous transfection of cells that allows scalability in producing transfected cells, for example in cell workflows that generate genetically modified cells.

In one aspect, provided herein is a continuous transfection system that includes, a culture vessel in sterile, fluid connection with a delivery line, and optionally a consumable assembly configured for use with the system. In some embodiments, the continuous transfection system of the disclosure includes one or more sensors, such as an optical sensor, a pressure sensor, an ultrasonic sensor, a thermal sensor, or any combination thereof. In some embodiments, a processor is electronically coupled to the system and operable to partially, or fully automate control of the system. For example, the processer includes functionality to automate control of one or more components of the system to monitor or change one or more conditions and/or processes for transfection, including flow rate of liquid into and/or through the system. In embodiments, the processor includes functionality to control flow through a first and/or a second input line, temperature of any contents contained within the system, such as a first and/or second reagent housing, manipulation of contents in a culture vessel, control of sensor(s) in the system, or any combination thereof.

In various embodiments, the cell culture vessel of the continuous transfection system of the disclosure is a bioreactor that optionally, includes a component configured to control the rate of mixing and/or stirring of particles in fluid, such as an impeller or an agitator, a shaker flask, bioreactor, and/or any other vessel capable of culturing cells. It will be appreciated that the capacity of the cell culture vessel generally varies. In some embodiments, the cell culture vessel has a maximum culture volume of greater than about 0.5 liters (L), including about 1 L, about 3 L, about 5 L, about 7 L, about 10 L, about 15 L, about 20 L, about 25 L, about 50 L, about 75 L, about 100 L, about 200 L, about 500 L, about 1,000 L or about 2,000 L.

In another aspect, the disclosure provides a consumable for use in the continuous transfection system of the disclosure. In some embodiments, the consumable includes a first and a second input line, configured for sterile, fluid connection to a first reagent housing and a second reagent housing, respectively, a mixing chamber that is fluidly connected to the at least first and second input lines, a delivery line fluidly connected to the mixing chamber, operable for sterile, fluid connection to a cell culture vessel, and a component configured to control flow rate of liquid into the first input line and/or the second input line, throughout the mixing chamber, and into the cell culture vessel. In some embodiments, the first reagent housing and the second reagent housing are in sterile, fluid connection with the first and second input lines.

In another aspect, the disclosure provides a consumable for use in the continuous transfection system. In some embodiments, the consumable includes at least a first input line and a second input line, configured for sterile, fluid connection to a first reagent housing and a second reagent housing, respectively, a mixing chamber that is fluidly connected to the at least first and second input lines, a delivery line fluidly connected to the mixing chamber, configured for sterile, fluid connection to a cell culture vessel, and a component configured to control flow rate of liquid into the at least two input lines, throughout the mixing chamber, and into the cell culture vessel. In some embodiments, the first reagent and second reagent housings may be in sterile, fluid connection with the first and second input lines.

It will be appreciated that the continuous transfection system, in various embodiments, may be configured in a variety of geometric arrangements such that, the continuous transfection system of the disclosure is a closed, sterile system that achieves continuous transfection of cells in a scalable manner. In some embodiments, the continuous transfection system includes at least or greater than about 2 input lines, e.g., between 2 and 10 input lines, such as between 2 and 4 input lines, 2 and 5 input lines, 2 and 6 input lines, 2 and 7 input lines, 2 and 8 input lines, or 2 and 9 input lines. Additionally, in some embodiments, a delivery line may be used in the system for the continuous introduction of a fluid, including transfection complexes, into a cell culture vessel as described herein. In various embodiments, the delivery line is any standard or specialized tubing size capable of facilitating continuous transfer of a fluid into and/or within the system. In some embodiments, a delivery tube has an inner diameter ranging from about 1 millimeter (about 0.04 inches) to about 20 millimeters (about 0.79 inches).

In yet another aspect, provided herein are methods for transfecting a population of cells with a payload, using the continuous transfection system described herein. The method includes providing a continuous transfection system that has a first reagent housing with a payload therein, wherein the first reagent housing is in fluid connection with a first input line, a second reagent housing with a transfection reagent therein, the second reagent being in fluid configuration with a second input line, and a fluidly connected, sterile, culture vessel containing a cell culture for transfection with the payload.

In various embodiments, the payload and the transfection reagent are continuously flowed into the first and second input lines, respectively, and then into a mixing chamber at a controlled rate to produce a transfection complex. In some embodiments, following mixing, the resulting transfection complex is transferred through the delivery line and into the cell culture vessel at a controlled rate and/or a fixed amount of time, including about 1 minute to about 24 hours, about 2 minutes to about 1 hour, about 5 minutes to about 30 minutes and about 7 minutes to about 20 minutes. In some embodiments, the amount of time from the formation of the transfection complex to the delivery of the transfection complex into the cell culture vessel is adjusted and/or tailored to best accommodate the transfection method and/or system of interest.

In embodiments, the payload for transfecting a population of cells within the system of the disclosure is any suitable payload known to the ordinarily skilled artisan and may be tailored according to the type of cell(s), assay or protocol, etc. In some embodiments, the payload is a nucleic acid (i.e., nucleotide, nucleoside, oligonucleotide, polynucleotide), a protein, a peptide, a polypeptide, or other biomolecule, or a combination thereof. For example, the payload can include a nucleic acid, such as DNA, RNA, or a component thereof (e.g., nucleotide, nucleobase, nucleoside, ribonucleotide, ribonucleobase, ribonucleoside or the like), as well as combinations thereof.

In various embodiments, a ribonucleoprotein complex is contained in the payload to be contacted with cells. In some embodiments, the cell culture is advantageously stirred or agitated during delivery of the transfection complex to the cell culture, so as to ensure continuous and even contact of the payload with the cell culture.

In various embodiments, the cell culture density varies depending, e.g., on the type of cells, protocols and/or assays of interest. In some embodiments, the cells of the cell culture are present at a density of between about 0.1×10⁶ cells/ml to about 1.0×10⁵ cells/ml, including about 0.1×10⁶ cells/ml to about 2.0×10⁶ cells/ml, about 0.5×10⁶ cells/ml to about 0.8×10⁵ cells/ml, about 1.0×10⁶ cells/ml to about 0.5×10⁵ cells/ml, and about 2.0×10⁶ cells/ml to about 4.0×10⁶ cells/ml, and about 2.5×10⁶ cells/ml to about 3.5×10⁶ cells/ml. In various embodiments, the cells are capable of undergoing transfection, such as mammalian cells, or components thereof, e.g, mitochondria. Embodiments of cells useful in the system and method of the disclosure include, mammalian cells, such as, by way of example Chinese hamster ovary (CHO) cells, human embryonic kidney (HEK) cells, peripheral blood mononuclear cells (PBMCs), T cells, large granular lymphocytes (LGL) or natural killer (NK) cells, B cells and combinations thereof.

### BRIEF DESCRIPTION OF THE DRAWINGS

For a fuller understanding of the nature and advantages of the present invention, reference should be made to the following detailed description taken in connection with the accompanying drawings. It is appreciated that these drawings depict only typical embodiments of the invention and are therefore not to be considered limiting of its scope. The disclosure will be described and explained with additional specificity and detail through the use of the accompanying drawings, in which:

FIG. 1 is a schematic depicting one embodiment of a system of the disclosure for batch transfection of cells;

FIG. 2 is a schematic depicting a consumable kit for use in a continuous transfection system in accordance with the present disclosure;

FIG. 3 is a schematic depicting the consumable kit of FIG. 2 in connection with the continuous transfection system in an embodiment of the present disclosure;

FIG. 4 is a schematic depicting the consumable kit of FIG. 2 in connection with the continuous transfection system in an embodiment of the present disclosure;

FIG. 5 is a schematic depicting the consumable kit of FIG. 2 in connection with the continuous transfection system in an embodiment of the present disclosure;

FIG. 6 is a schematic depicting the consumable kit of FIG. 2 in connection with the continuous transfection system in an embodiment of the present disclosure; and

FIG. 7 is a schematic depicting the consumable kit of FIG. 2 in connection with the continuous transfection system in an embodiment of the present disclosure.

### DETAILED DESCRIPTION

Before describing various embodiments of the present disclosure in detail, it is to be understood that this disclosure is not limited to the parameters of the particularly exemplified systems, methods, apparatus, products, processes, consumables, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. The terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified amount, dose, time, temperature, activity, level, number, frequency, percentage, dimension, size, amount, weight, position, length and the like. In instances in which the terms “about” and “approximate” are used in connection with the location or position of regions within a reference polynucleotide or polypeptide, these terms encompass variations of ±up to 20 base pairs or amino acid residues, ±up to 15 base pairs or amino acid residues, ±up to 10 base pairs or amino acid residues, ±up to 5 base pairs or amino acid residues, ±up to 4 base pairs or amino acid residues, ±up to 3 base pairs or amino acid residues, ±up to 2 base pairs or amino acid residues, or even ±1 base pair or amino acid residue.

The term “comprising” which is synonymous with “including,” “containing,” “having” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to an “inlet” includes one, two, or more inlets.

As used in the specification and appended claims, directional terms, such as “top,” “bottom,” “left,” “right,” “up,” “down,” “upper,” “lower,” “inner,” “outer,” “internal,” “external,” “interior,” “exterior,” “proximal,” “distal” and the like are used herein solely to indicate relative directions and are not otherwise intended to limit the scope of the disclosure or claims.

Where possible, like numbering of elements have been used in various figures. Furthermore, alternative configurations of a particular element may each include separate letters appended to the element number. Accordingly, an appended letter can be used to designate an alternative design, structure, function, implementation, and/or embodiment of an element or feature without an appended letter. For instance, an element “80” may be embodied in an alternative configuration and designated “80a.” Similarly, multiple instances of an element and or sub-elements of a parent element may each include separate letters appended to the element number. In each case, the element label may be used without an appended letter to generally refer to all instances of the element or any one of the alternative elements. Element labels including an appended letter can be used to refer to a specific instance of the element or to distinguish or draw attention to multiple uses of the element.

Various aspects of the present devices, systems, and methods may be illustrated with reference to one or more exemplary embodiments. As used herein, the term “embodiment” means “serving as an example, instance, or illustration,” and should not necessarily be construed as preferred or advantageous over other embodiments disclosed herein.

Various aspects of the present devices and systems may be illustrated by describing components that are coupled, attached, and/or joined together. As used herein, the terms “coupled”, “attached”, “connected” and/or “joined” are used to indicate either a direct connection between two components or, where appropriate, an indirect connection to one another through intervening or intermediate components. In contrast, when a component is referred to as being “directly coupled”, “directly attached”, “directly connected” and/or “directly joined” to another component, there are no intervening elements present.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

In various aspects, provided herein are systems, methods and consumables for the continuous transfection of cell cultures. In particular, the systems, methods and consumables provided herein advantageously allow for the continuous combination or mixing of transfection reagents, (e.g., lipid-based and/or polymer-based transfection reagents or the like) with a payload, e.g., to form a transfection complex. The systems described herein advantageously enable the payload and transfection reagent to complex for a fixed duration of time prior to delivery to the cell culture that is to be transfected with the payload (the target cell population, or target cell culture). Using the systems and methods provided herein, transfection complexes can be continuously generated and allowed to complex for a fixed period of time (i.e., an optimal time), hereinafter referred to as the “complexation time,” and continuously delivered to the target cell culture. Preferably, the target cell culture is continuously stirred or agitated as the transfection complexes are continuously added thereto, thereby ensuring that the transfection complexes are dispersed throughout the target cell culture, and minimizing the likelihood that the transfection complexes come into contact with only a subset of the target cells within the target cell culture (e.g., cells that are in close proximity to the delivery line).

The term “continuous transfection” is used herein to refer to a process whereby a payload and a transfection reagent are continuously mixed and, following mixture, continuously delivered to a population of cells. Preferably, the amount of time between mixing the payload and the transfection reagent until the mixture is delivered to the population of cells (the complexation time) is adjustable. For example, the complexation time can be fixed for the entire process of transfection of a target cell population with a desired payload. Alternatively, the complexation time can be adjusted throughout the transfection process.

The term “payload” refers to a biomolecule to be delivered into a cell (e.g., into the cytoplasm or nucleus of the cell). In various embodiments, a payload includes nucleic acids (e.g., DNA, RNA and combinations thereof), proteins, peptides and combinations thereof (e.g., ribonucleoproteins, and the like).

The term “transfection reagent” is well known in the art and refers to any chemical reagent or modifier capable of interacting and/or binding with nucleic acids and/or cell membranes to facilitate the uptake of the payload by cells. The ordinarily skilled artisan will readily appreciate that various types of transfection reagents are useful in the embodiments provided herein, including many commercially available transfection reagents. By way of example, in some embodiments, the transfection reagent is a composition that includes cationic or ionizable lipids, anionic polymers, or the like. In some embodiments, the transfection reagent is cationic-lipid transfection reagent, e.g., Lipofectamine® MessengerMAX™, Lipofectamine® 2000, Lipofectamine® 3000, used to increase the transfection efficiency of RNA (including mRNA and siRNA) or plasmid DNA into cell cultures by lipofection.

As used herein, the terms “peptide,” “polypeptide” and “protein” are used interchangeably to refer to a polymer of amino acid residues. The terms apply to naturally occurring amino acid polymers, as well as amino acid polymers in which one or more amino acid residue is an artificial chemical mimetic of a corresponding naturally occurring amino acid.

As used herein a peptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at 5′ (amino) terminus and a translation stop codon at 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refer to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The term “polynucleotide” or “oligonucleotide” or “nucleotide sequence” or “nucleic acid molecule” or “nucleic acid” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid.

Nucleic acids include but are not limited to genomic DNA, cDNA, mRNA, IRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid might be employed for introduction into, e.g., transfection of, cells, e.g., in the form of RNA which can be prepared by in vitro transcription from a DNA template. The RNA can moreover be modified before application by stabilizing sequences, capping, and polyadenylation. Generally, nucleic acid can be extracted, isolated, amplified, or analyzed by a variety of techniques such as those described by, e.g., Green and Sambrook, Molecular Cloning: A Laboratory Manual (Fourth Edition), Cold Spring Harbor Laboratory Press, Woodbury, NY 2,028 pages (2012).

It will also be appreciated that nucleic acids include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

In general, nucleotides contained in a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

In various embodiments, nucleic acids are RNA molecules that include oligonucleotides containing modifications. A variety of modification are known in the art and contemplated for use in the present invention. For example, oligonucleotides containing modified backbones or non-natural internucleoside linkages are contemplated. As used herein, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In various embodiments, modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Certain oligonucleotides having inverted polarity comprise a single 3′ to 3′ linkage at 3′-most internucleotide linkage i.e., a single inverted nucleoside residue which may be abasic (the nucleobase is missing or has a hydroxyl group in place thereof). Various salts, mixed salts and free acid forms are also included.

In some embodiments, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In embodiments, oligonucleotide mimetics are utilized in which nucleotide units are replaced with novel groups. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular, an aminoethylglycine backbone. The nucleobases are retained and are bound directly or indirectly to aza nitrogen atoms of the amide portion of the backbone. In various embodiments, oligonucleotides may include phosphorothioate backbones and oligonucleosides with heteroatom backbones. Modified oligonucleotides may also contain one or more substituted sugar moieties.

In some embodiments, nucleic acids include Locked Nucleic Acids (LNAs) to generate antisense nucleic acids having enhanced affinity and specificity for the target polynucleotide. LNAs are nucleic acid in which the 2′-hydroxyl group is linked to 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2.

In accordance with the disclosed technology, a plurality of cells comprising a “cell culture” or “culture of cells” is subjected to a transfection process, e.g., a transient transfection process. Examples of cells that may make up a cell culture include Chinese hamster ovary (CHO) cells, human embryonic kidney (HEK) cells, peripheral blood mononuclear cells (PBMCs), T cells, large granular lymphocytes (LGL) or natural killer (NK) cells, B cells and combinations thereof. Other non-limiting examples of cells used for transfection include, but are not limited to, a bacterial cell, a eukaryotic cell, a yeast cell, an insect cell, or a plant cell. In some embodiments, cells used for transfection are human embryonic kidney 293 (HEK293), E. coli, Bacillus, Streptomyces, Pichia pastoris, Salmonella typhimurium, Drosophila S2, Spodoptera SJ9, COS (e.g., COS-7), 3T3-F442A, HeLa, HUVEC, HUAEC, NIH 3T3, Jurkat, 293, 293H or 293F.

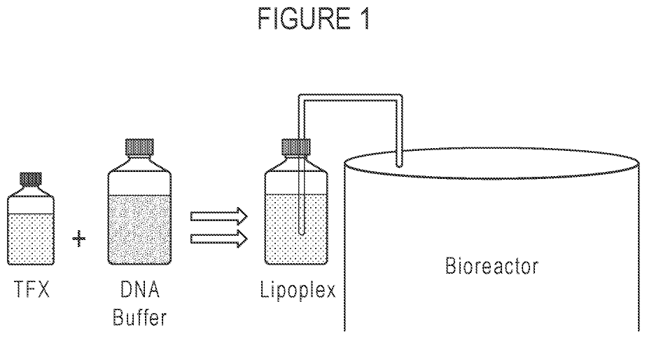

Turning to FIG. 1, shown is a “batch” method of transfecting target cell cultures, i.e., in the absence of a continuous transfection system as described herein. As shown in FIG. 1, the payload and transfection reagent are manually mixed to form a transfection complex in batch. The batch-produced transfection complex is then delivered to the target cell culture. As is clear from FIG. 1, batch transfection does not allow for (1) precise control of the complexation time (e.g., in cases wherein the transfection complex is delivered through a delivery line over time) and (2) delivery conditions that minimize local concentration effects of, e.g., delivery of a bolus of transfection complexes to a cell culture.

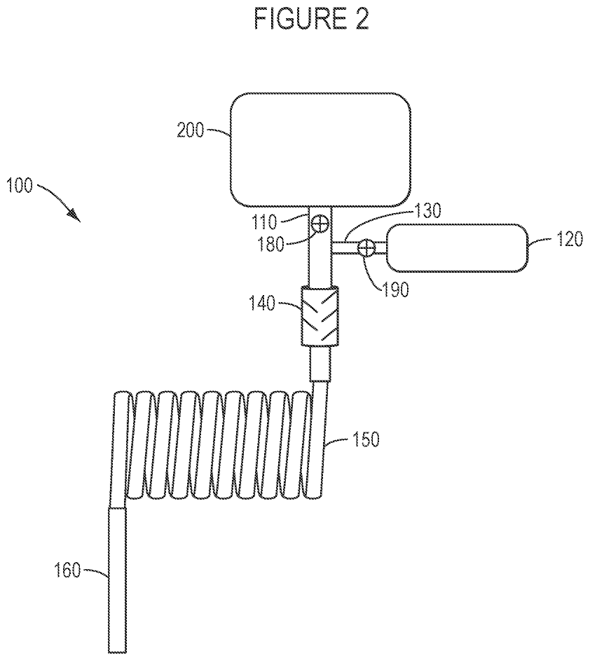

Turning to FIG. 2, shown is an embodiment of a consumable 100 that is used in the continuous transfection systems described herein. In some embodiments, the consumable shown in FIG. 2 includes both disposable and re-usable components and is configured for sterile connection to a culture vessel, to provide a closed, sterile continuous transfection system.

In various embodiments, the consumable 100 includes at least a first input line 110 and a second input line 130. The first and second input lines are adapted to be coupled to a first reagent housing 200 and a second reagent housing 120, respectively. For example, the first and second input lines are adapted to facilitate sterile connection to the first and second reagent housings. In embodiments, the first and second reagent housings include any format, e.g., bottles, tubes, bioprocess containers (e.g., bags), and the like.

The first and second input lines feed into a mixing chamber 140. As shown in FIG. 1, the contents of the first and second input lines intersect at a junction, and flow into the mixing chamber. The junction shown in FIGS. 1-7 is a T-junction, nevertheless, it will be understood that the junction can take on other configurations, e.g., a Y junction or the like.

Typically, the input lines are in sterile, fluid connection with the mixing chamber. Ideally, the mixing chamber 140 is a static or motionless mixer, i.e., it has no moving parts, and is configured to achieve maximal blending of solutions, causing the contents of the mixing chamber to travel from top to bottom. For example, is some embodiments, the mixing chamber is a static mixing chamber having an arrangement of baffles or other internal features that prevent a vortex effect and cause the contents of the mixing chamber to move from top to bottom. The ordinarily skilled artisan will readily appreciate that many types of mixing chambers are useful in the systems described herein and well-known to those skilled in the art, including but not limited to those described in U.S. Pat. No. 3,286,922, which is incorporated herein by reference in its entirety. Static mixers allow for continuous flow, thus the contents from the first reagent housing and the second reagent housing are in contact and can be carefully monitored and controlled. The degree of mixing can be controlled by varying the linear velocity or flow rate of the solution through the mixer, the type of mixer used, the diameter of the mixer and the number of features in the mixer.

The following parameters can be adjusted/tuned to ensure that the desired ratio of the first reagent (e.g., payload) and the second reagent (e.g., transfection reagent) are mixed together at the junction, and further that the flow of the transfection complex through the system for the desired amount of time prior to being transferred into a cell culture vessel:
a) volume of various components that constitute the fluid path of the continuous transfection system (e.g., height and area of the cross section of the input lines, mixing chamber, incubation chamber (if present); velocity of the flow of the first reagent (e.g., payload) and the second reagent (e.g., transfection reagent) into the junction; and
b) rate at which the fluid is flowed through the system (e.g., via one or more pumps).

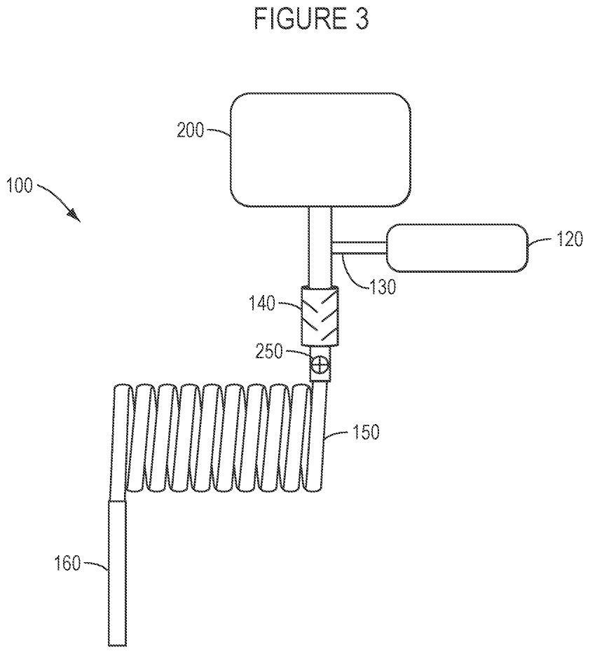

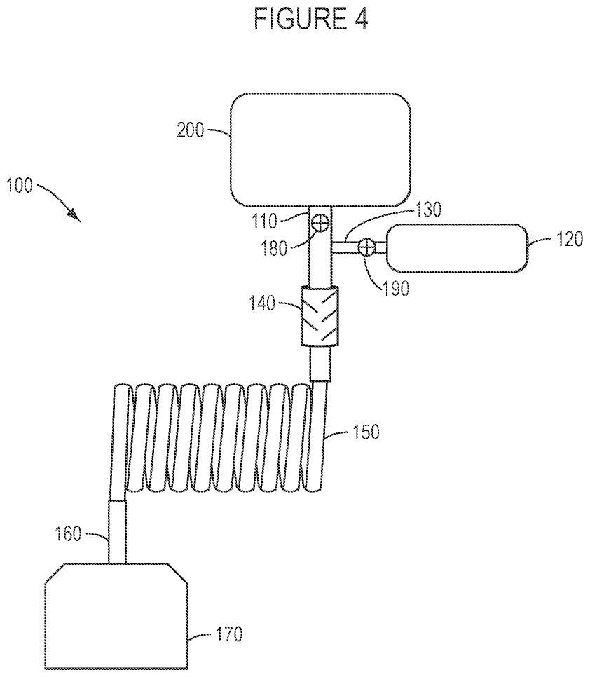

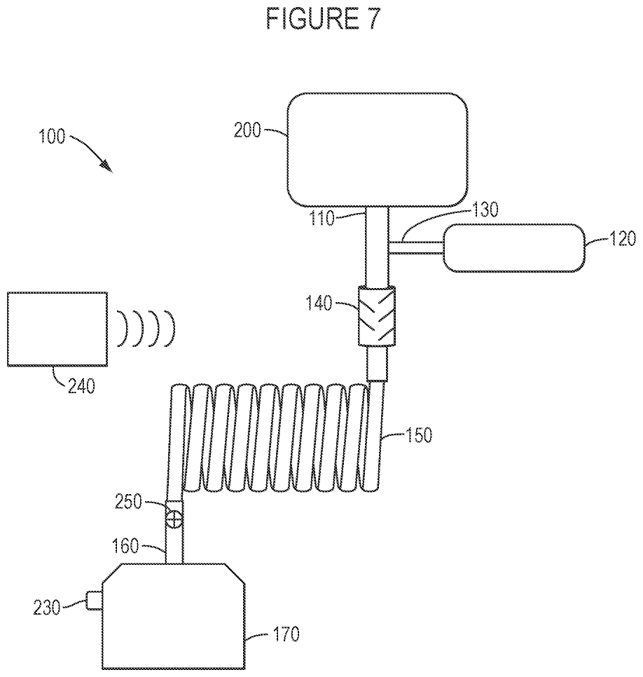

For example, is some embodiments, the ratio of the first reagent (e.g., payload) to the second reagent (transfection reagent) is adjusted by adjusting the rate at which a first pump and a second pump (e.g., as depicted in FIGS. 2 and 4) flow the first and second reagents, respectively into the system. Alternatively, in some embodiments, the ratio of the first reagent (e.g., payload) and the second reagent (e.g., transfection reagent) are adjusted by adjusting the dimensions of the first and second input lines, where the flow of the first reagent and the second reagent though the first and second input lines is controlled by a single pump (i.e., at the same rate), e.g., as depicted in FIGS. 3 and 7.

In some embodiments, the ratio of the first reagent to the second reagent is about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:500, about 1:1000, about 1:5000, about 1:10000, about 1:50000, about 1:100000 or about 1:1000000. In some embodiments, the ratio of the second reagent to the first reagent is about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:500, about 1:1000, about 1:5000, about 1:10000, about 1:50000, about 1:100000 or about 1:1000000.

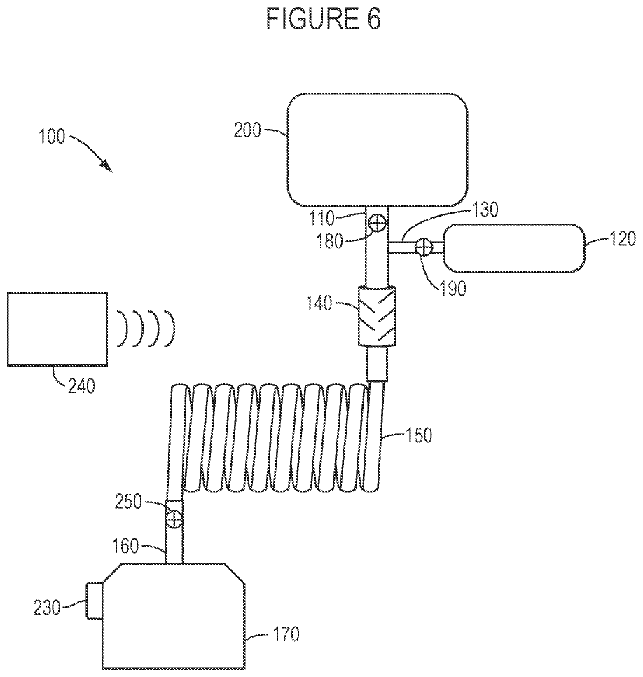

I will be appreciated that although FIGS. 2-7 depict two input lines, in some embodiments, the system includes one or more additional input lines, e.g., 1, 2, 3, 4, 5, 6, 7, 8, 9, 10, or more, input lines located either upstream from or downstream from mixing chamber 140. In embodiments, additional input lines are configured for sterile, fluid connection to additional reagent housings, waste containers, and the like. For example, the continuous transfection system shown in FIG. 6 includes input line 210 and associated reagent housing 220 located downstream from the mixing chamber and in fluid connection with delivery line 160. In some embodiments, additional input lines are used for the addition of buffers, media, or other desirable components to the transfection complexes prior to delivery to the target cell culture.

Mixing chamber 140 is fluidly connected to delivery line 160. FIG. 2 depicts a consumable that includes an incubation line 150 that is downstream of mixing chamber 140 and upstream from delivery line 160. The ordinarily skilled artisan will readily appreciate, however, that the incubation line is optional, and that some consumables may have a configuration wherein mixing chamber 140 is directly upstream of delivery line 160. The incubation line 150, when present, can be configured in a variety of ways. For example, the incubation line 150 depicted in FIGS. 2-7 has a coiled shape, but the incubation line could be interwoven, or in any other configuration.

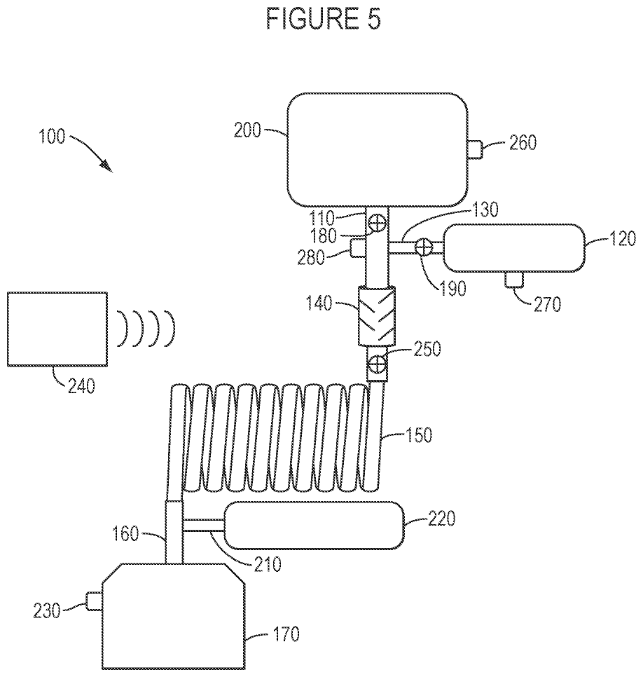

The continuous transfection system and consumables shown in FIGS. 2 and 4-7 include pumps 180 and 190, which are positioned along input lines 110 and 130, respectively, whereas the continuous transfection system and consumable shown in FIGS. 3 and 5 depict pump 250, positioned downstream of the mixing chamber (and optional incubation line), along the delivery line. Various types of pumps useful in continuous transfection systems described herein include, but are not limited to, peristaltic pumps, syringe pumps, diaphragm pumps, vacuum pumps, ultra-high vacuum pumps, capillary pumps, lift pumps, lobe pumps and the like.

As shown in FIG. 2, the continuous transfection system optionally includes an incubation line, which fluidly connects mixing chamber 140 to delivery line 160. Although the incubation line depicted in FIGS. 2 through 7 are represented in a coiled formation, the ordinarily skilled artisan will readily appreciate that many configurations/shapes of incubation (and input, and delivery) lines are suitable for use in the systems provided herein. In some embodiments, the incubation line has an interwoven configuration or a coiled configuration (e.g., as depicted in FIGS. 2-7).

The incubation line may have a length of from about 1 meter (about 3.28 feet) to about 15 meters (about 49.2 feet), including from about 5 meters (about 16.4 feet) to about 10 meters (about 38.2 feet). The incubation and delivery lines may have inner diameters ranging from about 1 millimeter (about 0.04 inches) to about 20 millimeters (about 0.79 inches).

In embodiments, the fluid path, e.g., including input lines, the mixing chamber, the incubation line (if present), the delivery line, and the like, is composed of materials (e.g., tubing) that ensure that fluids flow through the path uniformly and exhibit minimal fluid drag, thereby enabling precise control of the complexation time. Exemplary materials include polymers such as polypropylene, polycarbonate, rubber, silicone, cyclic olefin copolymers (COCs), cyclic olefin polymers (COPs), polydimethylsiloxane (PDMS), polystyrene, nylon, acrylic, high-density polyethylene (HDPE), low-density polyethylene (LDPE), polyolefins, and combinations thereof. Non-polymeric materials such as an alloy, metal, inorganic glass, or ceramic are also useful in the systems provided herein.

The components of the consumables and the continuous transfection system 100 provided herein are preferably made of materials that are non-reactive with biological materials (e.g., cell culture reagents, transfection reagents, payloads, and the like). In addition, the materials from which the various components are made are capable of withstanding sterilization, such as by autoclaving, irradiation, liquid chemical sterilization (LCS), plasma gas sterilization, vaporized hydrogen peroxide (VHP) sterilization or the like. Advantageously, the fluid path is made from materials that meet regulatory requirements for devices for the manufacture of cellular therapeutics.

Preferably, the consumable includes one or more sensors such as sensors 230, 260, 270 and/or 280 as depicted in FIG. 5. For example, the consumables and systems provided herein can include one or more sensors for monitoring and/or further controlling fluid flow through the systems. Sensors can include liquid sensors, analyte sensors, pH sensors, density sensors, pressure sensors, bubble detectors, thermal sensors, and the like. The ordinarily skilled artisan understands that one or more sensors may be positioned within the systems and associated consumables, e.g., along or within a transfer line, a reagent housing, a culture vessel, an incubation line, or the like.

The flow rate for fluid contents within the system of the disclosure, such as liquid media, transfection reagents, payload solutions and/or transfection complexes, can be controlled and manipulated by one or more pumps, e.g., one, two, three, four or more pumps, in operable connection with at least one component of the system. For example, the one or more pumps of the continuous transfection system can be one or more of syringe pump, a peristaltic pump, a diaphragm pump, a vacuum pump, an ultra-high vacuum pump, an aspiration pump, a capillary pump, a lift pump and a lobe pump, or any combination thereof, e.g., a peristaltic pump and a syringe pump. The pumps can be operated concurrently, for instance, to ensure 1) optimal complexation of a DNA/complexation buffer and transfection reagent for forming a transfection complex; and 2) continuous and efficient introduction of the transfection complex into the cell culture. For instance, a first pump can be configured to control the flow of one or more fluids through a first input line, while a second pump may be configured to control the flow of fluid through a second input line. Alternatively, a single pump is configured to control flow of one or more fluids through a first input line and second input line, wherein the first and second input lines are differently sized dimensionally to control fluid flow through the lines therefore controlling the amount of fluid from the first input line that contacts fluid of the second input line in a linear or exponential manner.

In embodiments, the consumables and systems provided herein include a processor such as processor 240 as shown in FIGS. 5-6. The processor can include hardware and software that controls the pump(s) and the flow rates of contents of the reagent housings and may be 1) directly connected to one or more components of the consumables through, e.g., cables, cords, and the like; 2) wirelessly connected, e.g., via cellular-based pairing to one or more components; or 3) connected both directly and wirelessly. As an example, the processor can provide a mechanism for automating the process according to pre-programmed parameters, and may include software components that can include automated collection of “run data” including, for example, the lot numbers of disposable components, temperature and volume measurements, cell number parameters, dose payload applied, complexation time, operator identity, date and time, target cell identity, and the like.

In some embodiments, the system of the disclosure includes a bar code-reading system to permit data entry of variables (for example, disposable set lot number and expiration date, lot number and expiration date of reagents, sample identifiers, and the like) into the device controller as part of the documentation of processing. This capability reduces the opportunity for data entry errors; as well as informing the system of any potential variations within lot number which may define structural variations in tube diameter.

In some embodiments, the consumables and systems provided herein include a processor and associated software providing functionality to prompt a user through the steps necessary for the proper insertion of tubing and other elements into the system. In some embodiments, software also includes functionality that initiates automated testing to confirm the correct insertion of tubing, reagent housings, cell culture vessels, and the like, as well as the absence of blockages, etc.

As shown in FIGS. 3-7, delivery line 160 is configured to be fluidly connected to culture vessel 170. Delivery line 160 has a proximal and a distal end, with openings at either side. The opening at the proximal end provides the point of attachment to the mixing line or alternatively incubation line, when present. The opening at the distal end is where the transfection complex exits the consumable portion of the continuous transfection system, and into the culture vessel. The delivery line can be positioned such that the opening at the distal end is at the surface or above the cell culture within the cell culture vessel. Alternatively, the delivery line can be positioned such that the opening at the distal end is within the cell culture in the cell culture vessel, e.g., midway down the culture, at the bottom of the cell culture/cell culture vessel, or the like.

It will be appreciated that a variety of culture vessels can be used in the continuous transfection systems described herein. For example, the culture vessel can be a tube, a flask, a plate (for adherent culture), a bioreactor (e.g., a stirred tank bioreactor), or the like. Various bioreactors useful in the systems provided herein include, but are not limited to, integrated single-use bioreactors, open-architecture single-use bioreactors, rocker bioreactors, and the like. By way of example, the culture vessels of the systems provided herein include, e.g., those described in U.S. Pat. Nos. 8,960,486; 9,943,814; 10,150,090; and 10,640,741; and U.S. Pat. App. Pub. No. 2019/0083947, each of which is hereby incorporated by reference in this disclosure in its entirety.

With reference to the system of FIG. 5, to allow for alterations to the path length and incubation time, in addition to the system 100 having a defined fluid path of the incubation line 150, a plurality of components of multiple systems (FIGS. 1-7) are connected to one another in series, e.g., multiple first and second reagent housings, coupled by a mixing chamber and leading into the incubation line 150. In some embodiments, at least 2, 3, 4, 5, 6, 7, 8, 9, 10 or more sets of first and second reagent housings coupled by a mixing chamber are fluidly connected upstream of the incubation line 150.

As discussed herein, it will be appreciated that incubation line 150 may be formed in a variety of geometric patterns, such as, by way of example, concentric coiled circles, ovals, ellipses and the like. In some embodiments, the incubation line 150 includes a tube geometry having any variety of turns, arcs, curves, and layers as shown in International PCT App. Pub. No.: WO 2020/076683, which is herein incorporated by reference in its entirety. In some embodiments, the incubation line 150 is shaped so as to avoid pooling and/or eddies within the flow path such that reagents within the flow path achieve a uniform particle flow to ensure a constant rate of particle mixing and flow rate to the culture vessel 170. In some embodiments, the incubation line 150 includes grooves, surface texturing and/or other surface modification(s) within the incubation line lumen to promote uniform particle flow. In some embodiments, the incubation line 150 includes grooves, surface texturing and/or other surface modification(s) within the incubation line lumen to inhibit uniform particle flow.

Advantageously, the continuous transfection systems described herein can be adjusted/tuned to ensure that the maximal number of cells are exposed to the transfection complex. In this regard, preferably, the culture vessel comprises a stirring element, such as an impeller, an agitator or a similar device. For example, in embodiments where the culture vessel is a stirred tank bioreactor, the speed at which the cells in the cell culture vessel are mixed (and by extension, the location of individual cells within the culture relative to the output of the delivery line throughout the process) can be tuned/adjusted so as to maximize the probability that all cells in the culture are evenly exposed to the transfection complex.

Various embodiments for stirring the contents of the culture vessels may be usefully employed in the continuous transfection systems disclosed herein. Non-limiting examples of stirring embodiments include, but are not limited to, those described in EP Pat. No. 245654, U.S. Pat. Nos. 7,628,528 and 10,092,787, each of which is hereby incorporated by reference in this disclosure in its entirety.

In additional aspects, the cell culture vessel comprises a maximum culture volume greater than about 0.5 liters (L), including but not limited to a maximum culture volume selected from the group consisting of about 1 L, about 3 L, about 5 L, about 7 L, about 10 L, about 15 L, about 20 L, about 25 L, about 50 L, about 75 L, about 100 L, about 200 L, about 500 L, about 1,000 L and about 2,000 L.

The system can further include various supports, housings, and the like for various components that can be coupled with the present system to perform methods and processes as described herein. For example, the system can include one or more support structures configured to hold and/or support various reagents, samples, filters, fluids, and the like. Support structures may include various hooks, hangers, and/or holders, as well as related implements known to the ordinarily skilled artisan. In some embodiments, the system includes a temperature control unit for regulating, or otherwise controlling the temperature of a fluid with the system, for example, within a culture vessel or incubation line.

The continuous transfection systems provided herein can further include a housing or cover that covers the entire system, or any component or combination of components thereof.

A delivery line, a tube and/or tubing may comprise an inner diameter ranging from about 1 millimeter (about 0.04 inches) to about 20 millimeters (about 0.79 inches). delivery line, a tube and/or tubing may comprise an inner diameter ranging from about 1 millimeter (about 0.04 inches) to about 20 millimeters (about 0.79 inches).

As previously noted, a major current limitation in molecular and cellular biology relates to the lack of reliable, scalable systems capable of accommodating complexation of a payload and a transfection reagent for a fixed period of time prior to delivery to the cell culture that is to be transfected with the payload. Accordingly, the systems disclosed herein advantageously accommodate such complexation and continuous delivery to a cell culture in, e.g., a transient transfection process. The disclosed systems and methods advantageously reduce culturing issues associated with incubation time variabilities and the discontinuous introduction of the complexed (payload/transfection reagent) solution to the culture-comprising vessel(s). Additionally, the systems described herein beneficially reduce or eliminate mixing variability with the cell culture vessel, thereby ensuring maximal and consistent cell exposure to the complexed solution. The systems also allow for minimal operator interaction and oversight during a large-scale process, thereby reducing the potential for adventitious contamination and/or processing difficulties often encountered with transient transfection.

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### EXAMPLES

The following examples describe improvements on cellular transfection methods and production of the resulting cells over those previously disclosed in the relevant art. The following examples are provided to demonstrate certain, non-limiting aspects of the disclosed technology.

### Example 1

### Bioreactor Based Continuous Transfection

The following example demonstrates the transfection of cells within a continuous transfection system as provided herein using a stirred tank bioreactor format.

A consumable for a continuous transfection system as described herein was connected to a 2 L bioreactor (Sartorius, Model: Biostat B 2 L Single, S/N: Project 4200047) to provide a closed, sterile, fluid path. Viral Production Cells (HEK-293-based) were grown in viral production medium to a viable cell density (VCD) of about 0.55×10⁶ cells/ml and introduced into the bioreactor. Throughout the experiment, the cells were cultured at 37° C., with continuous mixing at 200 rpm.

A GFP expression vector (pCDNA 3.1-based) was diluted in complexation buffer to a concentration of 1.5 μg/mL (“payload”), for about 10 minutes in a first reagent housing. The transfection reagent was provided in a second reagent housing. Simultaneously, the payload was pumped into the in-line static mixer via a standard syringe pump. The DNA/complexation buffer and transfection reagent were mixed to form a transfection complex using the in-line static mixer and subsequently flowed through an incubation line and delivery line at a rate of 12.97 mL/min. After 9 minutes had passed, the transfection complex passed through the outlet of the delivery line into the bioreactor. The payload and transfection reagents were pumped through the continuous transfection system for a total of 7.7 min, such that 100 mL of the transfection complex was ultimately delivered to the bioreactor.

Following delivery of the transfection complex, the cells were cultured for an additional 48 hours and subsequently harvested. GFP levels were then measured. The transfection efficiency was calculated to be about 83%, and the Mean Fluorescence Intensity (MFI) of the GFP was calculated to be about 260250 relative fluorescence units.

The foregoing demonstrates the ability to achieve continuous transfection using the systems and methods provided herein.

### Example 2

### Use of a Non-Continuous Transfection System in AAV Viral Particle Production

The following example demonstrates the ability to produce AAV particles using the systems provided herein.

Viral Production Cells (HEK-293-based) were grown in 2 L Viral Production Medium to a viable cell density (VCD) of about 0.55×10⁶ and introduced into a 5 L shake flask. Throughout the experiment, the cells were cultured at about 37° C., with continuous mixing at about 100 rpm.

An AAV-based GFP expression vector was diluted in complexation buffer (“payload”) to a concentration of about 1.5 μg/mL and incubated for about 10 minutes in a first reagent housing of a consumable as shown in FIG. 2. A transfection reagent was incubated for about 10 minutes in the second reagent housing. The payload and the transfection reagent were flowed into the in-line static mixer via a peristaltic pump and a standard syringe pump, respectively, to form a transfection complex. The transfection complex was flowed through the incubation line and delivery line at a rate of about 12.97 mL/min. The delivery line was inserted into a series of four 50 ml conical tubes to collect the transfection complexes. The payload and transfection reagents were pumped through the transfection system for a total of about 15.4 minutes, such that about 200 mL of the transfection complex pumped into the conical tubes (about 50 mL of payload and transfection reagents was transferred into each of the four 50-ml conical tubes). After the fourth conical tube was filled, the contents of the four tubes were simultaneously delivered to the cell culture in the shake flask.

The cells were subsequently cultured in the shake flask for about 20 additional hours at 37° C., and harvested. A portion of the cell culture was used to calculate the transfection efficiency, and a portion of the cells was used to calculate the AAV titer. The transfection efficiency was calculated to be about 63% (compared to a positive control), while the GFP intensity was calculated to be about 14500 relative fluorescence units. The AAV2 titer concentration was calculated to be about 5.4×10¹⁰ viral genome units per mL (vg/mL).

The foregoing additionally demonstrates the ability to achieve AAV production using the systems and methods provided herein.

### Example 3

### Shake Flask-Based Continuous Transfection/LV Particle Production

The following example demonstrates the transfection of cells and the production of lentiviral (LV) particles within a shake flask format for continuous transfection as provided herein.

A consumable for a continuous transfection system as described herein was connected to a 5 L shake flask. Viral Production Cells (HEK-293-based) were grown in LV-MAX™ production media (available from Thermo Fisher Scientific, Waltham, MA, USA) to a viable cell density (VCD) of about 1.0×10⁶ and introduced into the shake flask. Throughout the experiment, the cells were cultured at about 37° C., with continuous mixing at about 100 rpm.

The LV-MAX™ viral packaging mix (Thermo Fisher Scientific, cat. A43237) was diluted in complexation buffer to a concentration of about 2.5 μg/mL and incubated for about 10 minutes in a first reagent housing of a consumable as shown in FIG. 2. The DNA/complexation buffer (payload) solution was pumped into an in-line static mixer using a standard peristaltic pump. Simultaneously, the transfection reagent was pumped into the in-line static mixer via a standard syringe pump. The DNA/complexation buffer and transfection reagent were mixed to form a transfection complex using the in-line static mixer and subsequently flowed through an incubation line and delivery line at a rate of about 12.97 mL/min. on top of the cell culture in the 5 mL shake flask. The payload and transfection reagents were pumped through the continuous transfection systems for a total of about 15.4 minutes, such that about 200 mL of the transfection complex was directly delivered to cell culture within shake flask.

Cells were cultured for an additional 20 hours, at which time a first portion of the cell culture was removed and used to calculate the transfection efficiency, and a second portion of the cells was used to calculate the LV titer. The remaining cells were cultured for about 28 hours (i.e., 48 hours post-transfection). At this time, a portion of the cell culture was harvested and used to calculate live cell count (measured in units of live cells/mL). Another portion of the cell culture was harvested at 72 hours post-transfection and used to calculate infectious titer.

The transfection efficiency (as a function of % GFP expression compared to positive control) was calculated to be about 26%, and the live cell count concentration was determined to be about 35500 live cells/mL. In addition, the infectious titer concentration was calculated to be about 2.76×10⁶ transduction units per mL (TU/mL).

The foregoing further demonstrates the ability to achieve continuous transfection using the systems and methods provided herein.

It will be appreciated that systems, processes, and/or consumables according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, processes, products, and the like are not described herein in particular detail in order to avoid obscuring aspects of the example embodiments. Such aspects are, however, also contemplated herein.

While the instant disclosure provides certain illustrative aspects and describes the general principles of the described technology, those persons of ordinary skill in the relevant arts will appreciate that modifications in the arrangement and details of the disclosure may be introduced without departing from these aspects and principles. Accordingly, Applicant claims all modifications that are within the spirit and scope of the appended claims.

What is claimed is:

1. A continuous transfection system comprising:
a) at least a first input line and a second input line, wherein the first input line and the second input line are configured for sterile, fluid connection to a first reagent housing and a second reagent housing, respectively;
b) a mixing chamber that is fluidly connected to the at least first and second input lines;
c) a delivery line fluidly connected to the mixing chamber, wherein the delivery line is configured for sterile, fluid connection to a cell culture vessel; and
d) a component configured to control flow rate of liquid into the at least first and second input lines, throughout the mixing chamber, and into the cell culture vessel.

2. The continuous transfection system of claim 1, further comprising the first reagent housing and the second reagent housing in sterile, fluid connection with the first input line and the second input line.

3. The continuous transfection system of claim 1, further comprising the culture vessel, wherein the culture vessel is in sterile, fluid connection with the delivery line.

4. The continuous transfection system of any of claims 1 to 3, further comprising a sensor.

5. The continuous transfection system of claim 4, wherein the sensor is selected from the group consisting of: an optical sensor, a pressure sensor, an ultrasonic sensor, a thermal sensor and any combination thereof.

6. The continuous transfection system of any of claims 1 to 5, further comprising a processor, wherein the processor controls one or more conditions selected from the group consisting of: flow rate of liquid into the first input line and/or the second input line, temperature of any contents in the first reagent housing and/or the second reagent housing, any contents in the culture vessel, one or more sensors, and any combination thereof.

7. The continuous transfection system of any of claims 1 to 6, wherein the system is partially or fully automated.

8. The continuous transfection system of any of claims 1 to 7, wherein the cell culture vessel is a bioreactor.

9. The continuous transfection system of claim 8, wherein the bioreactor comprises a stirring element.

10. The continuous transfection system of claim 9, wherein the stirring element comprises an impeller or an agitator.

11. The continuous transfection system of any of claims 1 to 10, wherein the cell culture vessel comprises a maximum culture volume greater than about 0.5 liters (L).

12. The continuous transfection system of any of claims 1 to 11, wherein the cell culture vessel comprises a maximum culture volume selected from the group consisting of: about 1 L, about 3 L, about 5 L, about 7 L, about 10 L, about 15 L, about 20 L, about 25 L, about 50 L, about 75 L, about 100 L, about 200 L, about 500 L, about 1,000 L and about 2,000 L.

13. The continuous transfection system of any of claims 1 to 12, wherein the component configured to control the flow rate comprises a pump.

14. The continuous transfection system of claim 13, wherein the system comprises at least two pumps.

15. The continuous transfection system of claim 13, wherein the pump is selected from the group consisting of: a syringe pump, a peristaltic pump, a diaphragm pump, a vacuum pump, an ultra-high vacuum pump, an aspiration pump, a capillary pump, a lift pump and a lobe pump.

16. The continuous transfection system of claim 14, wherein the at least two pumps include a first pump that controls the flow of fluid through the first input line, and a second pump that controls the flow of fluid through the second input line.

17. The continuous transfection system of claim 13, wherein the pump includes a pump that controls the flow of fluid through the first input line, the second input line, or the first input line and the second input line.

18. The continuous transfection system of any of claims 1 to 17, wherein the system is a closed, sterile system.

19. The continuous transfection system of any of claims 1 to 18, wherein the system comprises between 2 and 10 input lines.

20. The continuous transfection system of claim 1, wherein the delivery line comprises an inner diameter ranging from about 1 millimeter (about 0.04 inches) to about 20 millimeters (about 0.79 inches).

21. A method for transfecting population of cells with a payload, comprising:
a) providing the continuous transfection system of any one of claims 1 to 20;
b) providing the first reagent housing comprising a payload, wherein the first reagent housing and the first input line are fluidly connected;
c) providing the second reagent housing comprising a transfection reagent, wherein the second reagent housing and the second input line are fluidly connected;
d) providing the culture vessel, wherein the culture vessel and the delivery line are fluidly connected, and wherein the culture vessel comprises a cell culture comprising the population of cells to be transfected with the payload;
e) continuously flowing the payload and the transfection reagent into the first and second input lines, and into the mixing chamber at a controlled rate, thereby producing a transfection complex; and
f) continuously flowing the transfection complex through the delivery line and into the cell culture vessel at a controlled rate;
wherein the amount of time from formation of the transfection complex to delivery of the transfection complex into the cell culture vessel is adjustable.

22. The method of claim 21, wherein the amount of time from the formation of the transfection complex to delivery of the transfection complex into the cell culture vessel is fixed.

23. The method of claim 21, wherein the payload is selected from the group consisting of a nucleic acid, a protein, a peptide, and any combination thereof.

24. The method of claim 21, wherein the nucleic acid is selected from the group consisting of a DNA, an RNA, or a combination thereof.

25. The method of claim 21, wherein the payload comprises a ribonucleoprotein complex.

26. The method of claim 22, wherein the fixed amount of time is in a range of about 1 minute to about 24 hours.

27. The method of claim 22, wherein the fixed amount of time is between about 2 minutes and about 1 hour.

28. The method of any of claims 21 to 27, wherein the cell culture in the cell culture vessel is stirred or agitated during delivery of the transfection complex to the cell culture.

29. The method of any of claims 21 to 28, wherein the culture vessel comprises a cell culture, and wherein the cells of the cell culture are present at a density between about 0.1×10⁶ cells/ml to about 2.0×10⁶ cells/ml.

30. The method of any of claims 21 to 29, wherein the cell culture vessel comprises a culture of cells selected from the group consisting of: Chinese hamster ovary (CHO) cells, human embryonic kidney (HEK) cells, peripheral blood mononuclear cells (PBMCs), T cells, large granular lymphocytes (LGL) or natural killer (NK) cells, B cells and any combination thereof.

31. The method of claim 30, wherein the culture of cells comprises T cells.

32. The method of claim 31, wherein the T cells are activated prior to contacting the cells with the mixture.

33. The method of claim 21, wherein the continuous transfection system comprises at least two pumps.

34. The method of claim 33, wherein the at least two pumps are operated concurrently or in series.
